(19) United States
(12) Patent Application Publication
(10) Pub. No.: US 2012/0209088 A1
(43) Pub. Date: Aug. 16, 2012

(54) CONTINUOUS NON-INTERFERING HEALTH MONITORING AND ALERT SYSTEM

(75) Inventor: Yoram Romem, Herzeliya (IL)

(73) Assignee: HEALTHWATCH LTD., Herzeliya (IL)

(21) Appl. No.: 13/499,588

(22) PCT Filed: Sep. 20, 2010
(86) PCT No.: PCT/IL10/00774

§ 371 Date: Apr. 25, 2012

Related U.S. Application Data
(60) Provisional application No. 61/246,990, filed on Sep. 30, 2009.

(51) Int. Cl. A61B 5/145; A61B 5/053; A61B 5/11

(52) U.S. Cl. 600/301; 600/309

(57) ABSTRACT

A seamless and preferably substantially continuous health monitoring system, designed for use by a healthy living being but also suitable for non-healthy living being, the system including a control module, a communication unit and one or more sensors. The sensors can be in-vivo nano-sensors, micro-sensors, subcutaneous, wearable or implanted sensors. The control unit includes an analysis subsystem having a processing unit and an alerting unit. Each of the sensors is configured to detect a predetermined physiological or chemical parameter of the living being. The communication unit is facilitated to transmit the detected parameters to the analysis subsystem. The processor analyzes the detected parameters to thereby determine if the health state of the monitored living being is abnormal. When at least one detected parameter or the health state is determined to be abnormal, the alerting unit is operatively activated to alert a predetermined alert receiving entity.

### CROSS REFERENCE TO RELATED APPLICATIONS

This application claims the benefit under 35 USC 119(e) from U.S. provisional application 61/246,990 filed Sep. 30, 2009, the disclosure of which is included herein by reference.

### FIELD OF THE INVENTION

The present invention relates to real-time health monitoring systems and more particularly, the present invention relates to a real-time health monitoring system, carried in-vivo or ex-vivo by a living-being being monitored, such that the system does not interfere with the everyday life of the monitored living being. Furthermore, the system of the present invention issues an alert upon detecting a potentially health hazardous situation.

### BACKGROUND OF THE INVENTION AND PRIOR ART

Various prior art systems can monitor health parameters of a person after the person has been hospitalized or detected as being in some health danger and therefore needs special monitoring attention. Prior art systems and methods require the monitored person to make some adjustments to his/her normal life style: going to a laboratory, wearing a special monitoring device, or undergoing routine or non-routine checkups. However, before being detected as being in danger, the person is considered to be a healthy person. As such, no disturbances to his/her normal life style are usually acceptable: the person expects to live without any materials and/or devices attached to his/her body or to allow any disruption to daily activities. The problem is that people (or any other living being), considered to be healthy, are not monitored and that leads to frequent situations, in which a person suddenly suffers from a fatal or significant medical problem event that causes a major change is his/her normal life, sometimes for the rest of his/her life. Early detection of such significant medical problems may prevent (or minimize) such events.

There is, therefore, a need and it would be advantageous to have a health monitoring system, implantable into and/or wearable by a living being to be monitored, wherein the system does not interfere with the everyday life of the monitored living being and issues an alert upon detecting a potentially health hazardous situation or a tendency to develop such situation. This “early warning” system is the subject of the current invention.

In a common method to reduce fatal/major health risks, a person undergoes annual health checkups to determine his/her health status. However, these checkups do not provide any assurance that a short time after the checkup, the health situation of the checked person will not deteriorate, nor do such checkups cover a significant percentage of health hazards that may cause a significant life style change for the person. It should be noted that in most populations, people do not conduct routine health checkups and therefore these populations are more exposed to health hazardous situations.

There is a need, therefore, that the health monitoring system will continuously check the well being of a person (or any other living being) that is considered healthy, covering a significant range of health hazards that might cause a significant life style change/limitation, and provide an alert as early as possible—all this with no significant limitation to the normal life style of the person bearing the system. Preferably, with no limitation, no special routine action required of the monitored living being in order to get the alert, no special surgery should be needed for the system nor should limiting wearable devices be needed, as the system's major goal is to let the person continue his/her normal life until a potentially dangerous health situation occurs or starts to develop. Naturally, such a system may also be used by a sick person, detecting potential deterioration situations or new problems.

The term “continuous monitoring”, as used herein with conjunction with a health monitoring system, refers to a health monitoring system, facilitated to monitor a living being substantially continuous, day and night, when the monitored living being is awake or asleep, and active in substantially all common activities of such living being.

The term “seamless”, as used herein with conjunction with a wearable or implantable device, refers to a device that when worn by a person, the device puts no significant limitation to the normal life style of that person. Preferably, with no limitation, the in-vivo sensors insertion process does not require surgery or painful injection, but rather can be inserted by a laser-based insertion technique, an injection by micro-needles or by nano-needles, by swallowing pills or capsules, by transdermal patches, transmembrane receptors, using RF-based techniques or other nano sensors insertion means known in the art. Furthermore, no activity is required from the monitored person in order to be able to be alerted when needed.

Prior art monitoring systems are not seamless, but rather interfere with the life style of the system wearer or require some special active actions by the wearer in order to get an alert. Prior art systems are not capable of detecting chemical parameters, for example in the blood or other internal systems (which require in-vivo sensors or methods to extract body fluids or organs). Furthermore, prior art systems are usually not adaptive to the specific person's dynamic health state, and do not algorithmically integrate between chemical and physical inputs to generate a reliable personal local alert. For example, U.S. Pat. No. 6,840,904, given to Jason Goldberg, provides a portable device that receives data provided by one or more wearable sensors and displays statistical data related to the sensor data or otherwise related to the person. The system further includes a remote computer. FIG. 2 of U.S. Pat. No. 6,840,904, illustrates a portable medical monitoring device and sensors coupled to the device, wherein the portable device is mounted on the person's wrist and is wirely connected to a sensor mounted on the person's finger.

The term “nano sensor”, as used herein, refers to a device that is constructed using molecular manufacturing techniques, also referred to as nano-technology.

The terms “nano technology based sensor” and “nano sensor” are used herein interchangeably.

The term “abnormal”, as used herein with conjunction with health related parameters, refers to a parameter value or one or more ranges of values which are defined as health hazardous and require attention. For example, the normal blood pressure of an adult person is in the range 80-120 mm Hg. Typically, a blood pressure of 130 mm Hg would not be considered hazardous. However, if a person has a stable blood pressure around 85±10, and suddenly it goes to 125±10, this may be considered as an abnormal case. The threshold value from which the high blood pressure parameter is considered as health hazardous may vary and can be set personally and optionally, dynamically updated, either manually or automatically, by an adaptation algorithm. Once the high blood pressure parameter, in the above example, is set, any value out of the set threshold value will then be considered as abnormal for that person.

It would be further advantageous for the health monitoring system to have an algorithm that combines several parameters in the decision whether an abnormal situation occurs. Referring to the hereabove blood pressure example, when the blood pressure is proximal to the threshold value and another parameter, such as arrhythmia, is proximal to its abnormal state, the combination of both may suffice to trigger an alert.

### BRIEF SUMMARY OF THE INVENTION

The principal intentions of the present invention include providing systems for monitoring multiple aspects of the health of a living being and to issue an alert upon detecting a potentially health hazardous situation. The system is designed to be used by healthy living being and thereby, the system does not interfere with the everyday life of the monitored living beings. It should be noted that the system does not require any operative activity by the living being (or from anyone else) in order to provide the alert when needed. It should be noted that the system is not limited for use by healthy living beings and can be also used by non-healthy living beings. Furthermore, since a healthy living being may object to a surgical procedure or to a painful injection, there is an optional transport (delivery) method, preferably non-surgical injectionless intake and transport, and maintenance provisions for the in-vivo components of the system.

According to the teachings of the present invention, there is provided a seamless and preferably substantially continuous health monitoring system, designed for use by a healthy living being but also suitable for non-healthy living being. The system includes a control module, a communication unit and one or more sensors and preferably motion-posture detectors. The control unit includes an analysis subsystem having a processing unit, preferably memory and an alerting unit.

Each of the one or more sensors is configured to detect a predetermined physiological or chemical parameter (including of parameters of internal systems such as the blood vessels, kidneys, lung, etc.) of the monitored living being, preferably a person. The communication unit is facilitated to receive the detected parameters from each of the one or more sensors and transmit the detected parameters to the analysis subsystem. The processor of the analysis subsystem of the control module analyzes the detected parameters to thereby determine if one or more detected parameters or a combination of the detected parameters are abnormal, preferably in conjunction with the determined motion-posture state of the monitored living being. When at least one of the detected parameters is determined to be abnormal, or the combination of various parameters (which may not individually be abnormal) constitutes an abnormal situation, alerting unit is operatively activated to alert one or more predetermined alert receiving entities.

Preferably, the processor has an optional adaptation algorithm that determines the “normal state” of the individual living being, so that the thresholds and other parameters' characteristics are individually set. Furthermore, the adaptation mechanism is substantially continuously active, so that the dynamic nature of the human state is taken into account in the adaptation process. Furthermore, the processor can determine the ergometric parameters and physical status of the monitored living being (standing, lying down, extreme activity etc.), using motion-posture detectors controlled by the system control unit.

Preferably, the analysis subsystem includes a memory for storing at least a portion of the detected parameters. The stored detected parameters are used for trends analysis, for adaptation analysis and for enabling data extraction for further external analysis. The analysis subsystem analyzes the trends to detect abnormal trends in the detected parameters or a combination thereof, and wherein, when at least one of the trends is determined to be abnormal, the alerting unit is operatively activated to alert one or more predetermined alert receiving entities.

In variations of the present invention, the definition of the abnormality of the physiological or chemical parameter may be personally adaptive. In variations of the present invention, the definition of the abnormality is dynamically adaptable per the changing state over time of the living being and optionally, the motion-posture status.

Optionally, the processing unit of the analysis subsystem analyzes and determines the correlation between the detected parameters of two or more of the detected, thereby creating correlated parameters. When the detected correlated parameters are determined to be abnormal, the alerting unit is operatively activated to alert one or more predetermined alert receiving entities. Preferably, the system is facilitated to provide the alert with no operative action performed by the living being during health monitoring.

The control module can be a wearable module or an in-vivo module.

The sensor can be a wearable sensor or an implanted/in-vivo sensor.

The sensor can be delivered to an in-vivo target location, via blood vessels, via the digestive system or via the respiration system. In variations of the present invention, the sensor is delivered to a subcutaneous location. Optionally, the sensor is implanted during a non related surgery.

The communication unit includes one or more communication subunits, wherein each sensor is coupled with one or more of the communication subunits.

In variations of the present invention, the sensor is a nano-sensor or a micro-sensor. Preferably, the sensor is delivered to an in-vivo target location, in a non-painful procedure such as laser-based insertion, RF-based technology, by swallowing pills or capsules, by trans-dermal delivery patches or by injection using micro-needles or nano-needles.

In variations of the present invention, the sensor is delivered to an in-vivo target location, using targeted-liposome delivery techniques. In other variations of the present invention, the sensor is delivered to an in-vivo target location, using nanotube delivery techniques.

Optionally, the sensor requires no internal power source. Optionally, the communication unit or a communication subunit are configured to transmit a signal to the nano sensor and to receive a modulated echo of the transmitted signal, returned from the nano sensor, and wherein the modulated echo carries the information sensed by the nano sensor.

The wearable control unit is optionally a wearable device selected from the group including a wrist watch, a patch, an earring, a necklace, a bracelet and an armlet. In variations of the present invention, the wearable control unit is a wearable device attached to or integrated into a mobile electronic device, or a wheel chair or a personal device carried regularly by the living being. The mobile electronic device can be a cellular phone, a PDA, a wearable indicator or a mobile PC.

In variations of the present invention, the control module includes an oral control unit, disposed in the oral cavity of the living being. The oral control unit can be disposed inside one or more natural or artificial teeth of the living being.

The oral control unit may further include an internal power source and a maintenance unit, wherein the maintenance unit facilitates external maintenance activities of the oral control unit. The external maintenance activities are selected from a group of maintenance activities including updating the processing unit, setting up and update of parameters, downloading data, inserting a new or a replacement nano sensor, recharging internal power source, and performing diagnostic processes of selected members of the health monitoring system.

Preferably, the maintenance unit includes storage for nano sensors, and wherein the maintenance unit is facilitated to deliver the nano sensor on demand or by a predetermined time interval.

Optionally, the oral control unit further includes an oral test unit having at least one oral sensor and at least one oral sampler. The maintenance unit preferably includes storage for test analysis elements and storage for waste resulting from tests analysis. The at least one oral sampler collects oral material selected from the group of orally disposed materials including oral fluids, breath and blood. The oral test unit is operatively activated to engage one or more of the analysis elements with the one or more oral materials, thereby producing a testable material. The at least one oral sensor is configured to sense the testable material and create tested data. The maintenance unit transfers the tested data to the analysis subsystem of the control module. Preferably, the one or more test analysis elements are reagents, wherein the reagents are part of the oral sensor.

In variations of the present invention, the maintenance unit further includes a loading service channel for loading the storage for test analysis elements from an external source, wherein the loading service channel is sealingly closed during normal operation. Preferably, a loading device operatively loads test analysis elements through the loading service channel to the storage for test analysis elements. Preferably, the maintenance unit further includes a disposing service channel for disposing accumulated waste from the storage for waste to an external location, wherein the disposing service channel is sealingly closed during normal operation, wherein preferably, a disposal collecting device operatively removes waste from the storage for waste through the disposing service channel. The above mentioned loading and disposal are optionally made by a dental professional.

In variations of the present invention, the sensor is a digital acoustic sensor facilitated to sense bodily acoustic data, wherein the bodily acoustic data includes heart beat, lung and breathing sounds.

In variations of the present invention, the sensor is selected from the group of physical sensors including an electric sensor, an optical sensor, an acceleration sensor (usually an accelerometer for each of the three dimensions), a pressure based sensor, a conductivity sensor and a humidity sensor.

Optionally, the physical sensors sense bodily movement related data, wherein the definition of the abnormality depends also on the bodily movement related data.

Optionally, the physical sensors sense bodily posture related data, wherein the definition of the abnormality depends also on the bodily posture related data.

Optionally, the movement related data and the bodily posture related data are processed by a motion-posture detection unit.

Typically, the control module further includes an internal power source. Preferably, in in-vivo control units, the internal power source is a micro-battery or a nano-battery.

An aspect of the present invention is to provide a method for monitoring the health status of a living being. The method includes a step of providing a seamless health monitoring system, which system includes a control module including a control module having an analysis subsystem having a processing unit, and an alerting unit. The control module further includes communication unit and one or more sensors.

The method further includes a step of sensing designated health related parameter by the one or more sensors, thereby generating sensed data, transmitting the sensed data to the communication center, transmitting the sensed data to the analysis center, analyzing the sensed data, and determining if the sensed data is abnormal. Upon determining that the sensed data is abnormal, the method proceeds with selecting a proper alert type, transmitting the selected alert type(s) to the alerting unit, and activating the alerting unit.

In variations of the present invention, the method further includes the steps of analyzing the sensed data by the processing unit, thereby generating analyzed sensed data, and determining whether the analyzed sensed data is abnormal. Upon determining that the analyzed sensed data is abnormal, the method proceeds with selecting a proper alert type, transmitting the selected alert type(s) to the alerting unit, and activating the alerting unit.

In variations of the present invention, the control module includes an oral control unit, disposed in the oral cavity of the living being, wherein the oral control unit further includes a maintenance unit, wherein the seamless health monitoring system further includes an oral test unit having at least one oral sensor, and at least one oral sampler, and wherein the method further includes the steps of collecting selected oral materials from the oral cavity of the living being, transferring the oral materials to the test unit, transferring test analysis elements from an optional storage for test analysis elements or from external sources to the test unit, engaging the analysis elements with the oral materials, thereby producing a testable material, sensing the testable material by the at least one oral sensor, thereby creating sensed data, and proceeding with step of transmitting the sensed data to the analysis center.

In variations of the present invention, the seamless health monitoring system further includes a waste disposal storage, and wherein the method further includes the step of disposing waste materials resulted from the engagement of the analysis elements with the oral materials.

### BRIEF DESCRIPTION OF THE DRAWINGS

The present invention will become fully understood from the detailed description given herein below and the accompanying drawings, which are given by way of illustration and example only and thus not limitative of the present invention:

FIG. 1 is a schematic block diagram of a health monitoring and alert system, according to embodiments of the present invention, having a wearable control unit;

FIG. 2a illustrates, by way of example, an elongated nanotube for delivering nano technology based sensors, according to embodiments of the present invention;

FIG. 2b illustrates, by way of example, a spherical nanotube for delivering nano technology based sensors, according to embodiments of the present invention;

FIG. 3 illustrates, by way of example, a liposome for delivering nano technology based sensors, according to embodiments of the present invention;

FIG. 4 is a schematic block diagram of a health monitoring and alert system, according to embodiments of the present invention, having an oral control unit;

FIG. 5 is a schematic illustration of a health monitoring and alert system, as shown in FIG. 4, whereas the oral device is implanted in a tooth;

FIG. 6 illustrates the main components of a health monitoring and alert system, according to embodiments of the present invention, as disposed on a human being;

FIG. 7 is a schematic flow diagram that outlines the steps of monitoring the health status of a living being, performed for example on the system shown in FIG. 1 or 2, and the steps of activating an alerting unit upon detecting a potentially health hazardous situation; and

FIG. 8 is a schematic flow diagram that outlines a cycle of monitoring the health status of a living being.

### DETAILED DESCRIPTION OF THE INVENTION

The principal intentions of the present invention include providing systems for monitoring multiple aspects of the health of a living being and to issue an alert upon detecting a potentially health hazardous situation. The system is designed to be used by healthy living being and thereby, the system does not interfere with the everyday life of the monitored living beings and does not require any operative action from the monitored living being in order to provide the alert when needed. It should be noted that the system is not limited for use by healthy living beings and can be also used by non-healthy living beings. Furthermore, since a healthy living being may object to a surgical procedure or to a painful injection, there are optional non-surgical injectionless transport and maintenance provisions for the in-vivo components of the system.

The present invention will now be described more fully hereinafter with reference to the accompanying drawings, in which preferred embodiments of the invention are shown. This invention may, however, be embodied in many different forms and should not be construed as limited to the embodiments set forth herein; rather, these embodiments are provided, so that this disclosure will be thorough and complete, and will fully convey the scope of the invention to those skilled in the art. Unless otherwise defined, all technical and scientific terms used herein have the same meaning as commonly understood by one of ordinary skill in the art to which this invention belongs. The methods and examples provided herein are illustrative only and not intended to be limiting.

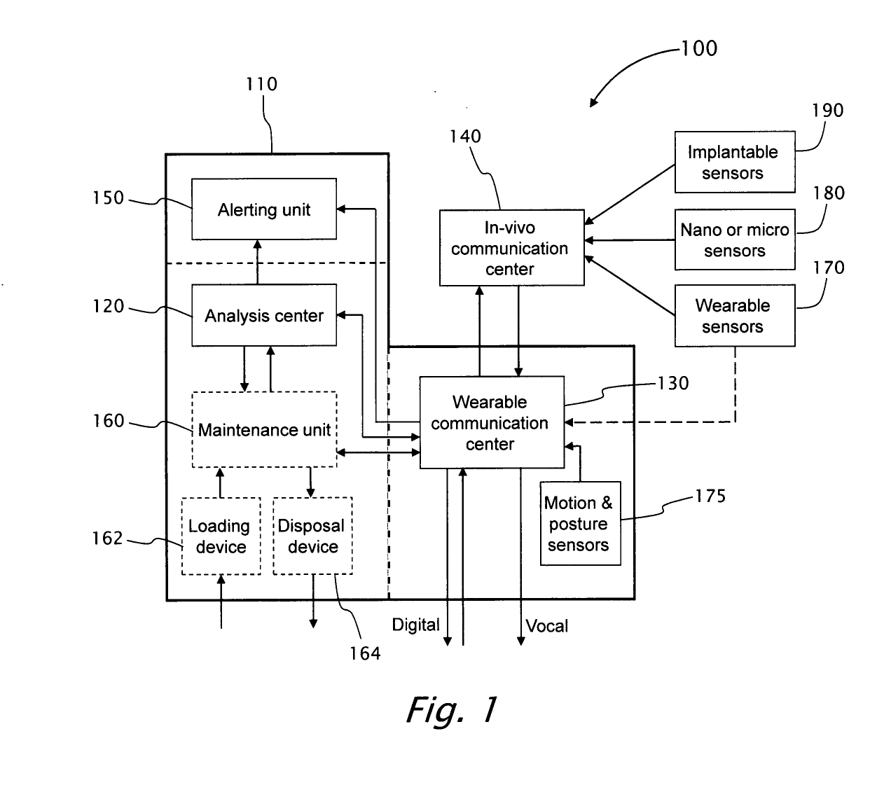

Reference now made to the drawings. FIG. 1 is a schematic block diagram of a seamless and preferably continuous health monitoring and alert system 100, according to embodiments of the present invention, having a wearable control unit 110. System 100 further includes one or more sensors selected from the group consisting of nano or micro sensors 180, implantable sensors 190 and wearable sensors 170. Wearable control unit 110 includes an analysis center 120, a communication center 130, an alerting unit 150 and optionally, a maintenance unit 160. System 100 may further include an in-vivo or ex-vivo communication center 140.

Analysis center 120 receives health status data from all the sensors (170, 180, 190), through communication center 130 and optionally, through in-vivo communication center 140. Optionally, analysis center 120 receives posture orientation and bodily motion data from motion and posture sensors 175, such as accelerometer based sensors and orientation sensors. Motion and posture sensors 175 may sense, for example, movement related data enabling the system determine instantaneous body states such as running, jumping, exerting physical force, etc. Motion and posture sensors 175 may further sense, for example, posture orientation such as standing, lying, sitting, etc. In variations of the present invention, the sensed bodily movement related data and sensed bodily posture related data are processed by a separate motion-posture detection unit (not shown).

Analysis center 120 includes a processing unit that analyzes the health status data received from the sensors (170, 180, 190), and thereby determines if a health hazardous situation occurs. The processing unit may further calculate values, compare thresholds, trends, averages etc, and may provide the calculated data to an external recipient. Preferably, analysis center 120 further includes memory for storing data for calculations, comparisons to past measurements, determining trends, calibration, determining sensors reliability, further remote analysis at external places and for future use (for example, for use in physical exercise consulting).

In variations of the present invention, the definition of the abnormality of the physiological or chemical parameter is personally adaptive, wherein the “normal” health state of a particular monitored living being is personally set. In variations of the present invention, the definition of the abnormality is dynamically adaptable per the changing state over time of the living being.

Upon detecting abnormal health related parameters, or an abnormal state determined as a result from an analysis of combined inputs acquired from different sensors, or from a trends analysis, analysis center 120 sends alert information to alerting unit 150. Optionally or additionally, analysis center 120 sends alert information to communication center 130 that sends alert information to a predetermined external recipient. Optionally, the processing unit of analysis center 120 analyzes and determines the correlation between the detected parameters of two or more of the detected, thereby creating correlated parameters. When the detected correlated parameters are determined to be abnormal, the alerting unit is operatively activated to alert one or more predetermined alert receiving entities.

Alerting unit 150 is activated by analysis center 120. Alerting unit 150 may have various alerting types, such as the following non limiting examples: vocal, visual and digital signals designated for various target recipients (typically, via communication center 130). In variations of the present invention, alerting unit 150 is a separate unit and not integrated into wearable control unit 110.

Communication center 130 receives input data from the sensors (170, 180, 190) and optionally from motion and posture sensors 175, and transfers the data to analysis center 120. Communication center 130 receives alerts requests from alerting unit 150 and communicates the alerts to various predetermined external devices such as, with no limitations, a cellular phone, a PDA, a computer, wearable indicator, etc. Communication center 130 may also receive maintenance requests from maintenance unit 160 and transmit the maintenance requests to various predetermined external remote devices. Communication center 130 may also receive requests for additional data information, such as sensed data, from an external source and transfer the requests to analysis center 120, which in turn can activate and/or interrogate the appropriate sensor (170, 180, 190). Communication center 130 may also receive maintenance/setup data information from an external source and transfer the maintenance/setup data information to maintenance unit 160. In variations of the present invention, communication center 130 is a separate unit and not integrated into wearable control unit 110.

System 100 uses one or more sensors to detect a potentially health hazardous situation when occurs. The sensors types are selected from the group consisting of nano sensors and/or micro-sensors 180, implantable sensors 190 and wearable sensors 170.

Implantable sensors 190 can be any implantable sensing device facilitated to sense in-vivo events, gathering data and send indications. It should be noted that in the context of the present invention, which prefers not to use surgery procedures, if the monitored living being undergoes a surgery anyhow, it is possible to add implantable sensor/s without violating the above preference. For example, U.S. Pat. No. 5,411,535, given to Tadashi Fujii et al., provides a cardiac pacemaker improved to reduce the weight and size and to unburden the wearer of the pacemaker, while ensuring safe wireless transmission of signals. The cardiac pacemaker includes a main body having at least two electrodes for detecting cardio-information, a control section for performing a control by outputting pulses on the basis of the cardio-information, and a transmitting section for modulating the pulses and transmitting the modulated pulses. The pacemaker also has pacing electrode portion having a receiving section for receiving the transmitted pulses and stimulating electrodes activated by the pulses output from the receiving section.

Wearable sensors 170 can be any wearable device, having means for sensing ex-vivo and in-vivo events, gathering data and transmit data to analysis center 120. Wearable sensors 170 are optional, as wearable sensors 170 must not interfere in the life style of the human or animal wearing wearable sensors 170. For example, U.S. Pat. No. 6,413,223 given to Boo-ho Yang et al., provides a device for noninvasive, continuous monitoring of arterial blood pressure for advanced cardiovascular diagnoses.

Nano sensors 180 are constructed using molecular manufacturing techniques, also referred to as nano technology. Nano (or micro) sensors 180 can be a chemical sensor operating in the blood system, digestive system, kidneys sub-system or any other biological system. Nano (or micro) sensors 180 can be a pathogen detector, tissue chemical imbalance detector, cancerous cells detector, etc. in variations of the present invention, sensors 180 are encapsulated in nano-capsules and nano-spheres, mainly for transporting, targeting and increase survivability. The nano sensors 180 are used continuously to detect and measure needed parameters such as HDL/LDL cholesterol, Hemoglobin, Hematocrit, Triglyceride, glucose, HbA1c, Bilirubin, Creatinine, PSA, CEA, Calcium Cerum, CPK, Lymphocytes, Monocytes, Basophils, Neutrophils, Eosinophils, Myelocytes, Blood pressure, Heart beat (pulse), Blood count, Body temperature, Oxygen saturation, Sweat, Conductivity etc. The nano sensors 180 are continuously available for transmitting the sensed data (usually by responding to an interrogation pulse) either directly to communication center 130 or indirectly, via in-vivo communication center 140.

A nano sensor 180 can be inserted by a laser-based insertion technique, an injection by micro-needles or by nano-needles (which does not yield a painful experience by the person as a regular injection does, thus maintaining the preferred objective of “injectionless goal”, in the context of painful injections), by swallowing pills or capsules, by transdermal patches, transmembrane receptors, using RF-based techniques or other nano sensors insertion means known in the art. Some of the above techniques are usually used for different purposes (for example, TransPharma's RF-MicroChannels® for drug delivery are created within milliseconds, with no resulting skin trauma or pain. Alma Lasers' cosmetics treatments with the Pixel® CO2, laser light passes through the patented Pixel® micro optics lens array to penetrate the skin with tiny thermal channels), however, the current invention is using similar techniques for sensors intake.

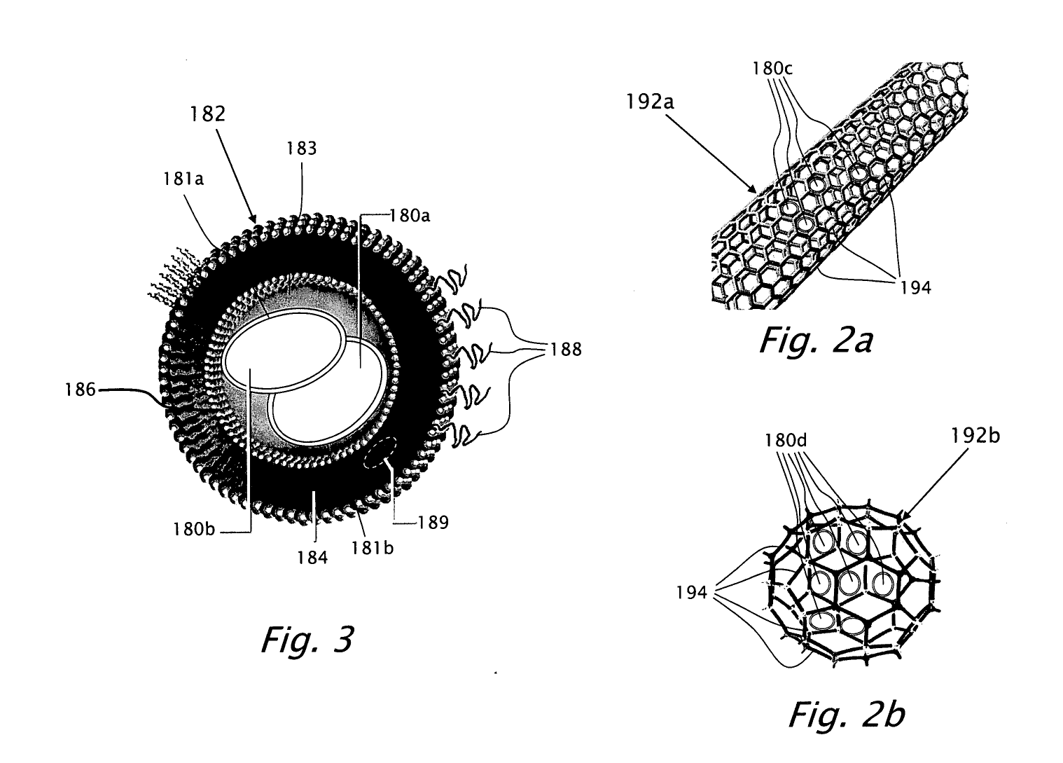

Reference is made to FIGS. 2a and 2b, which illustrate by way of example, nanotubes 192 adapted to deliver nano sensors 180c and 180c, according to embodiments of the present invention. FIGS. 2a illustrates a portion of an elongated nanotube 192a and FIGS. 2b illustrates a portion of a spherical nanotube 192b. Nanotubes 192 are used to deliver an active matter such as nano or micro sensors 180c and 180d to target organs through the skin. Optionally, Nano-needles are used to penetrate cells for carbon nanotubes 192 sensor delivery (for example, UK-based researchers from the School of Pharmacy, University of London, found that they were able to enter a variety of cell types, including human cancer cells). Optionally, health monitoring and alert system 100 uses this technique for sensor delivery. By piercing plasma membranes and heading straight to the cytoplasm, the functionalized carbon nanotube encounters fewer biological barriers and delivers the sensor more directly. The nanotubes also offer a structural advantage being extremely thin but very long, thereby offering a large surface area on which to graft the required sensor. This technique facilitates regulation of the amount of sensors loaded onto the nanotube.

It should be noted that often nano sensors 182 undergo a chemical reaction upon sensing a designated parameter. Such nano sensors 182 include an active region, often referred to the “reagent”, which reagent undergoes a chemical reaction upon sensing a designated matter. Therefore, such a deformed nano sensor 182 needs to be replaced. Carbon nanotubes 192 are facilitated to deliver a multiplicity of nano sensors 182 to thereby create a reservoir of nano sensors 182.

FIG. 3 illustrates, by way of example, a liposome 182 adapted to deliver nano sensors 180a and 180b, according to embodiments of the present invention. Liposome 182 is used to deliver an active matter such as nano or micro sensors 180a and 180b to target organs via the blood system, using homing peptide 188.

Liposome 182 encapsulates/entraps the one or more nano/micro sensors 180 inside a region 183 of aqueous solution enclosed, by a hydrophobic membrane 186, which can be dissolved into the cell membrane. Sensors 180a and 180b include an external layer 181a and 181b respectively, which external layer is lipid-soluble, configured to fuse with a lipid bilayer 184 of liposome 182 and other bilayers, such as the cell membrane, passing through the lipids and thereby delivering nano sensors 180. For example, item 189 is shown passing through lipid bilayer 184.

Nano sensors 180 are used to monitor a target organ or any other in-vivo biological target. For example, in-vivo transportation can be performed by carbon nanotubes (CNTs), which are extremely narrow, hollow cylinders made of carbon atoms (may include Titanium dioxide for enhanced Osseointegration or other means). Optionally, health monitoring and alert system 100 uses time-resolved Raman spectroscopy to monitor and detect nano sensors 180 moving through the blood circulation system.

Nano sensors 180 are positioned at a designated target location by various transport and targeting methods, for example, through the blood, homing peptide 188 (see FIG. 3) or any other biological species attached as a ligand to the liposome in order to enable binding via a specific expression on the targeted delivery site of nano sensor 180. The targeting ligand can be a monoclonal antibody (making an immunoliposome or membrane recognition and adhesion molecules), a vitamin or specific an antigen, thus creating a “targeted liposome” acting as vehicles or carriers. Therefore, a targeted liposome can target nearly any cell type in a living body. It should be noted that liposome 182 is typically protected from the host body immune system by a protective layer 189. It should also be noted that using nano-tubes 192 rather than liposomes 182, facilitates leveraging the durability characteristics, which enables extending the interval periods between sensors' replacements.

Nano sensor 180 is kept at a target designated location/site, for example, by adhesion or adsorption. An alert is generated and transmitted to analysis center 120 when nano sensor 180 is dispatched from the target designated location/site. Biomarkers are adhered to the surface of the liposome (when used as the vehicle) which delivers it to the target. They are controlled by a diffusion (mobility) mechanism, as explained hereinbelow, and monitored by the analysis center 120.

Nano sensor 180 can sense a location shift, for example by associated biomarkers; comparing the current location with the original stable location. The location shift (which is typically a mobility change indication) is transmitted to analysis center 120. For example, transmission is embodied using pulsed gradient spin echo NMR techniques. The pulsed gradient spin echo NMR technique for such alert requires either dual components for each nano sensor 180 (one acting as a location indicator and the second is the nano sensor itself), or the assessment of the nano sensors 180 self mobility from the self diffusion coefficient. The mobility corresponds to the relaxation effect that determines the diffusivity of the molecule. Several nano sensors 180 can use the same biomarker as a “mobility reference”.

Nano sensor 180 can transmit the sensed data either as 1/0 states or as scaled values or by other conventional transmission methods. Typically, the process of transmission has two phases: firstly, communication center 130 sends an interrogation/inquiry pulse to a specific nano sensor 180; then, the specific nano sensor 180 acts as an electric capacitor and responds with the same pulse modulated by the “sensed data” the specific nano sensor 180 contains; the response is received by communication center 130, which communication center 130 sends to analysis center 120 for decoding. Typically, the same transmission method provides alerts for a location shift of the nano sensor 180, discussed hereabove.

Analysis center 120 receives indications regarding the location of nano sensor 180 and determines whether an alert should be issued, a location-correction is needed or whether a certain nano sensor 180 stopped functioning (typically, both require a sensor replacement procedure. It should be noted that typically, a replacement of a nano sensor is a simple delivery of a new nano sensor, wherein when using nanotubes 192, the old nano tube 192 is withdrawn). Accordingly, analysis center 120 indicates to alerting unit 150 the alert type needed.

In variations of the present invention, the sensor (170, 175, 180, 190) is a digital acoustic sensor facilitated to sense bodily acoustic data, wherein the bodily acoustic data includes heart beat, lung and breathing sounds.

In variations of the present invention, the sensor (170, 175, 180, 190) is selected from the group of physical sensors including an electric sensor, an optical sensor, an acceleration sensor (usually an accelerometer for each of the three dimensions), a pressure based sensor, a conductivity sensor and a humidity sensor.

It should be noted that to minimize the living being's inconvenience and limitations, wearable control unit 110 can be worn as a wrist watch, a patch, an earring, a necklace, a bracelet, an armlet, etc. Optionally, wearable control unit 110 can be customized per wearer's preferences, for non-interference in his/her daily life. Maintenance unit 160 is used mainly to setup and update various parameters of health monitoring and alert system 100, facilitating data extraction from the memory and to perform self-testing of various sub-systems of health monitoring and alert system 100. Wearable control unit 110 may be attached to or integrated into a mobile electronic device or a wheel chair or other devices that are continuously situated proximal to the living being.

It should be further noted that communication center 130 and optional in-vivo communication center 140 can be used together, sharing the communication tasks in any configuration. Typically, a wearable sensor 170 communicates with communication center 130, residing in wearable control unit 110.

It should be further noted that some of the processing tasks can be performed at a remote monitoring center. The communication center 130 can optionally send the data to any remote processor, which can further process the information, compare the obtained data to corresponding data obtained from other monitored people, make statistics-based decisions and other decision-making issues to improve alerts (for example by detecting suspicious trends that did not trigger the automatic alert but a physician may want to further check the person) and providing information for assisting the treatment of the living being once getting to a treating facility.

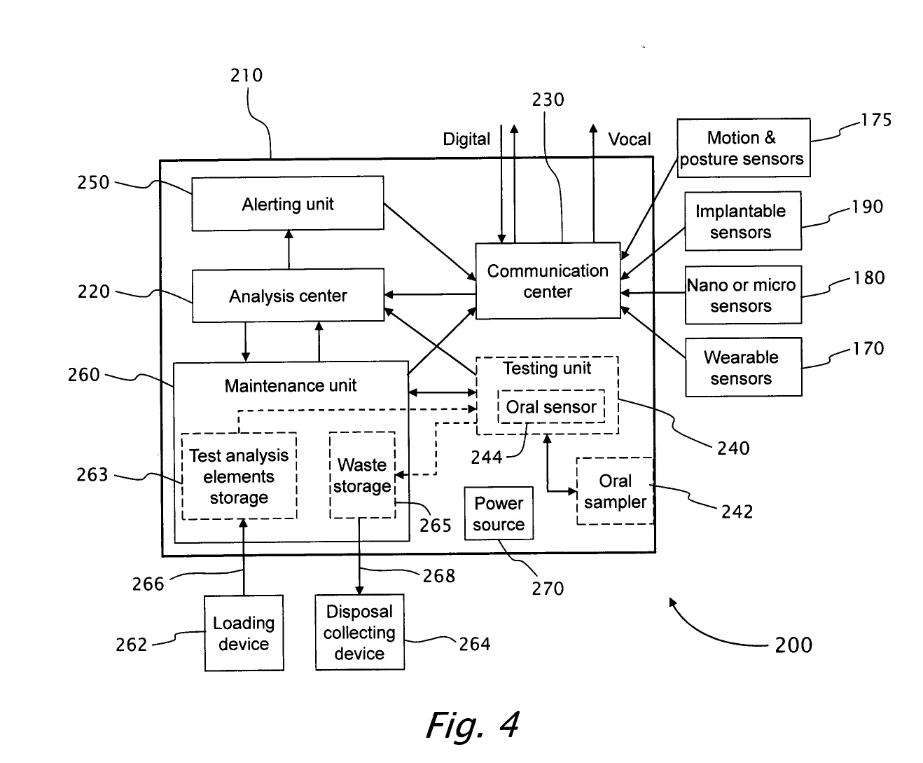

In variations of the present invention, a portion of the health monitoring and alert system is disposed orally, into one or more teeth. Reference is now made to FIG. 4, which is a schematic block diagram of a health monitoring and alert system 200, having an oral control unit 210. Generally, health monitoring and alert system 200 operates similarly to health monitoring and alert system 100, except for a few adaptations. Health monitoring and alert system 200 does not need to be worn by the monitored living being, and therefore does not interfere with the everyday life of the monitored living being. Furthermore, health monitoring and alert system 200 has direct contact with some in-vivo entities of the monitored living being, such as saliva and exhalation.

Analysis center 220 includes a processing unit that analyzes the health status data received from the sensors (170, 175, 180, 190), and thereby determines if a health hazardous situation occurs. The processing unit may further calculate values, compare thresholds, perform comparisons to past measurements, determine trends, determine sensors reliability, perform calibrations, compute averages, create adaptive normal states references etc, and may provide the calculated data to an external recipient. Preferably, analysis center 220 further includes memory for storing data for calculations and for future use (for example, when calculating trends).

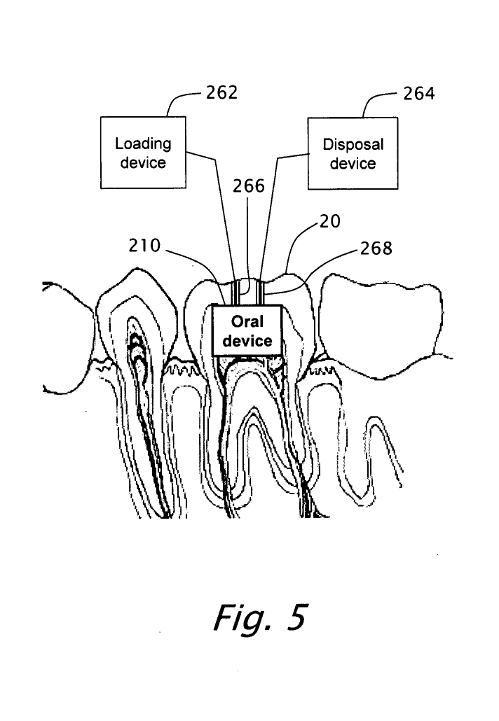

Reference is also made to FIG. 5, which is a schematic illustration of health monitoring and alert system 200, whereas oral control unit 210 is disposed inside one or more natural or artificial teeth 20. System 200 further includes one or more sensors selected from the group consisting of nano and/or micro sensors 180, implantable sensors 190 and wearable sensors 170. Oral control unit 210 includes an analysis center 220, a communication center 230, an alerting unit 250, preferably a maintenance unit 260 and optionally, a test unit 240. Analysis center 220 and communication center 230 are generally similar to respective units 120 and 130 of health monitoring and alert system 100.

Test unit 240 resides in oral control unit 210 and serves as a small laboratory to perform chemical tests. Test unit 240 is operatively coupled with one or more oral samplers 242, configured to collect oral material selected from the group of orally disposed materials including oral fluids such as saliva, blood and other available oral materials like breath. Test unit 240 receives the samples of materials from sampler 242, as well as analysis elements (lab materials), needed for specific tests, directly from a loading device 262 (via service channel 266, see FIGS. 4 and 3), or indirectly from test analysis elements storage 263 of maintenance unit 260. Test unit 240 is operatively activated to engage the one or more of reagents with the one or more sampled oral materials, thereby producing a testable material. The reagent is typically part of an oral sensor 244, configured to sense the testable material and create test results. Sometimes, oral sensor 244 needs to be replaced upon detection of a target sensed matter, which chemically reacts with the reagent, but usually, only the reagent is replaced by the next one from the reservoir.

Test unit 240 reports the test results to analysis center 220 for further analysis. The disposal from the tests is returned by test unit 240 either directly to a disposal collecting device 264 (via service channel 268, see FIG. 3), or indirectly to a waste storage 265 of maintenance unit 260.

Maintenance unit 260 (and optionally, maintenance unit 160 of health monitoring and alert system 100) has various functions in maintaining the orderly operation of health monitoring and alert system 200. Maintenance unit 260 is configured to:
a) add needed chemical reagents for testing unit 240;
b) insert a new (or a replacement of) nano/micro sensor 180;
c) re-charge power source 270 of oral control unit 210;
d) dispose of waste material through disposal collecting device 264;
e) setup and update various parameters of health monitoring and alert system 200;
f) perform diagnostic processes of selected members of health monitoring and alert system 200; and
g) download stored data.

It should be noted that typically, oral control unit 210 is installed inside an artificial tooth 20 by a dentist. In case of a need for replacement or maintenance, it is done by a dental treatment. Preferably, oral control unit 210 includes disposal unit 262 for disposing of chemical waste trough a special gate in channel 268, which gate opens only during maintenance.

It should also be noted that the fact that the transmission of nano sensors 180 is “passive” (i.e. just returning inquiry pulses from communication center 130) enables system 100 to get the required power from oral control unit 210 or wearable control unit 110 only.

It should be further noted that oral control unit 210 is typically powered by micro-batteries or nano-batteries. Optionally, the shelf-life of the batteries can be increased by nano-particles coated on the surface of the electrode. Part of the recharging method is performed by the normal operational motion of the oral control unit 210. For example, regular daily food chewing and/or daily teeth brushing can cause the batteries to recharge. Typically, the wearable control unit 110 is also powered by micro-batteries. However, the recharging of the batteries is performed by regular movement, and only the replacement of a battery of the oral control unit requires a dentist visit. A special alert is sent by the maintenance unit when a battery is about to require replacement.

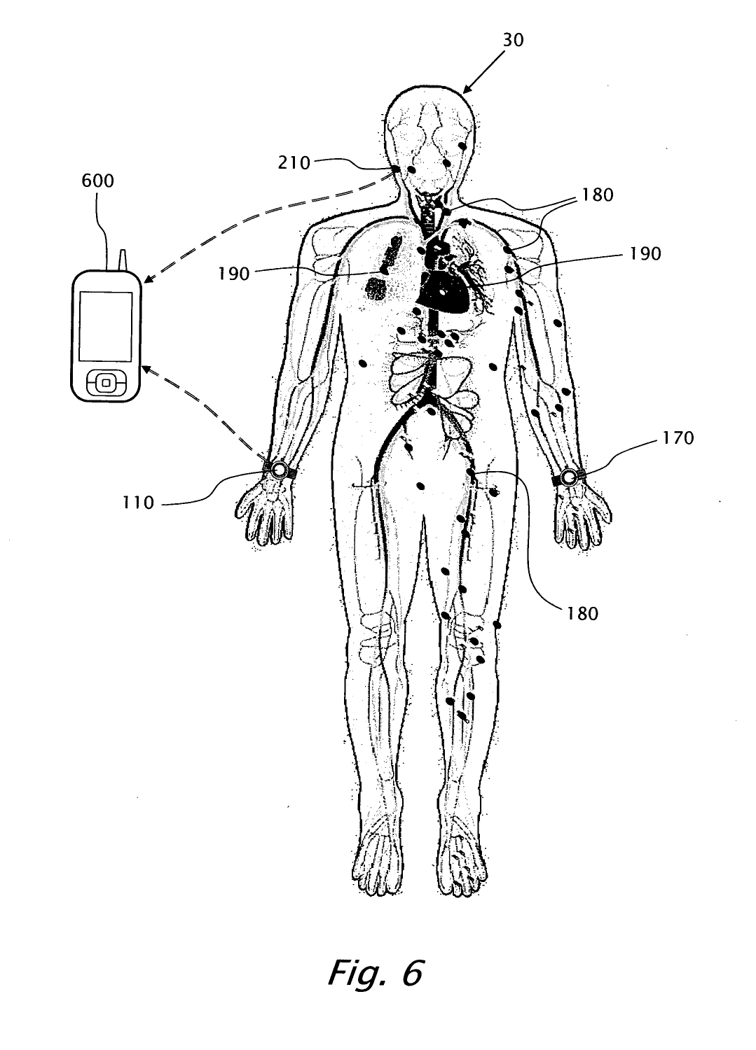

Reference is also made to FIG. 6, which illustrates, by way of example, the main components of a health monitoring and alert system (100, 200), according to embodiments of the present invention, as disposed on a human being 30. With no limitation, wearable control unit 110 is shown being implemented as a wrist watch and alternatively, oral control unit 210 is embodied in a tooth. Wearable sensor 170 is also shown being implemented as a wrist watch. Nano sensors 180 are disposed at various locations in the blood system, the lymphatic system, the digestive system, the urinary system etc.—per the specific configuration of the individual health monitoring and alert system. Implantable sensors 190 are shown monitoring the hearty and the lungs. When a potentially health hazardous situation occurs, an alert is activated as well as an alert message is sent to an external receiving device 600 such as a mobile phone (or any device with communication). Optionally, the recipient of the alert message returns an acknowledge message, for example, in the form of an SMS message. Upon receiving the acknowledge message, the control unit (110, 210) deactivated the alert (to be resent after an interval per the configuration of the system). Other configurations might include another living being and/or living beings and/or a multiple of remote monitoring centers.

In variations of the present invention, the control unit (110, 210) is facilitated to communicate with other implantable devices that maybe implanted in the living being, for example a pacemaker.

Optionally, the health monitoring and alert system (100, 200) includes sensors for detecting the characteristics of the physical activities of the living being, for example, acceleration sensors, pressure sensors, orientation sensors, etc.

An aspect of the present invention includes providing a method for monitoring the health status of a living being and issuing an alert upon detecting a potentially health hazardous situation.

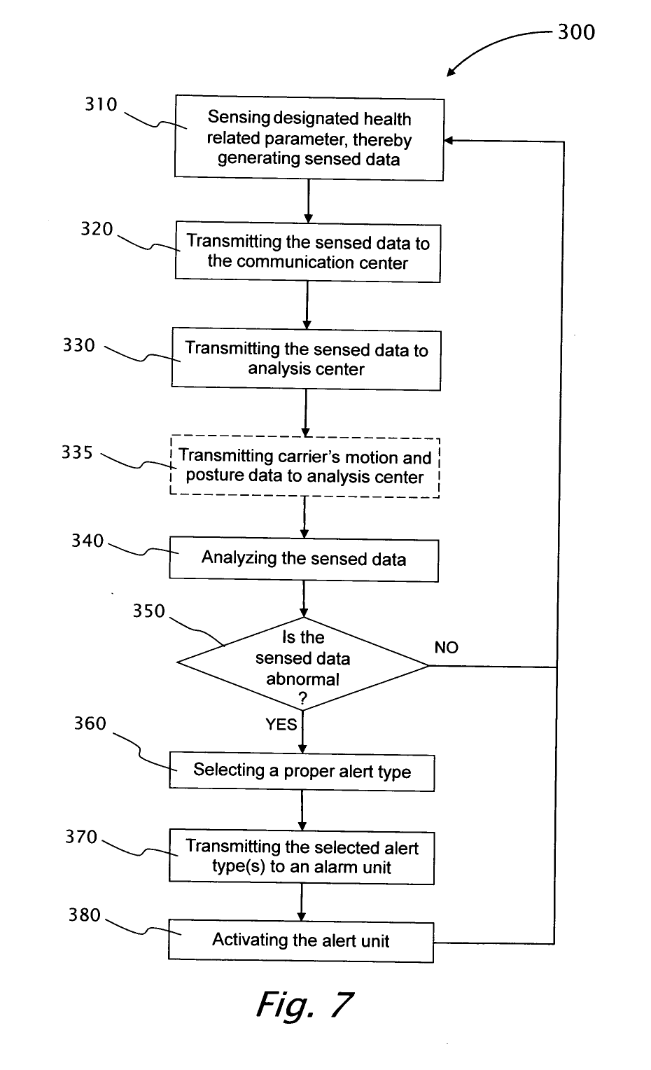

Reference is made to FIG. 7, which is a schematic flow diagram 300 that outlines the steps of monitoring the health status of a living being, performed for example on system 100 or 200, and the steps of activating an alerting unit (150 or 250, respectively) upon detecting a potentially health hazardous situation. Method 300 includes the following steps:
Step 310: sensing designated health related parameter, thereby generating sensed data.
   Each sensorᵢ (170, 180 or 190) senses the parameter that sensorᵢ is designed to measure, and thereby generating sensed data. For example, sensorᵢ is a nano sensor 180 designed to measure the level of Triglycerides in the blood. Hence, in this example, the sensed data is the measured level of Triglycerides in the blood.
Step 320: transmitting the sensed data to the communication center (130, 140 or 230).
   The sensed data is transmitted to communication center (130, 140 or 230). To continue the example, the measured level of Triglycerides in the blood is transmitted to communication center (130, 140 or 230).
Step 330: transmitting the sensed data to the analysis center (120 or 220).
   The sensed data is transmitted to the analysis center (120 or 220). To continue the example, the measured level of Triglycerides in the blood is transmitted to the analysis center (120 or 220).
Step 335: transmitting carrier's motion and posture data to the analysis center (120 or 220).
   Optionally, the sensed motion and posture data is transmitted to the analysis center (120 or 220). For example, movement related data can be running jumping, exerting physical force, etc.; posture orientation can be standing, lying, sitting, etc. it should be noted that the motion and posture data is used as input to the analysis algorithm for the determination of the appropriate thresholds to determine an abnormal state.
Step 340: analyzing the sensed data.
   The sensed data is analyzed by the processing unit of the analysis center (120 or 220). To continue the example, the level of Triglycerides in the blood is analyzed. For example, the processing unit calculates:
      IF {Triglycerides level}<10 mg/dl;
         AND sufficient time elapsed since last identical state identified;
      THEN send an alert type j to the alert unit (150 or 250);
      ELSE
      IF {Triglycerides level}>250 mg/dl;
         AND sufficient time elapsed since last identical state identified;
      THEN send an alert type k to the alert unit (150 or 250).
Step 350: determining if the sensed data is abnormal.
   If the sensed data is within the normal range, go to step 310.
   It should be noted that a preliminary step of determining the “normal range” (“normal range” being non-abnormal range) for a specific individual (function of parameters like age, family history, life style etc.) and a specific ergometric state (such as standing, lying, extreme effort, etc.) is optionally performed. Optionally, the parameters and coefficients are remotely set up and/or controlled.
Step 360: selecting a proper alert type.
   It has been determined that the sensed data is abnormal. To continue the example, an alert type j or k is set.
Step 370: transmitting the selected alert type(s) to the alerting unit (150 or 250).
   The selected alert type is transmitted to the alerting unit (150 or 250).
Step 380: activating the alerting unit (150 or 250).
   Alert unit (150 or 250) is activated with the selected alert type. To continue the example, the alert type activating a vocal ‘beep’ and sending an SMS message to a predetermined phone number.
   Go to step 310.
(end of steps details of process 300)

Optionally, method 300 further includes the steps of: sending an acknowledge message by the recipient of the alert message, for example, in the form of an SMS message; and upon receiving the acknowledge message, the control unit (110, 210) deactivates the alert.

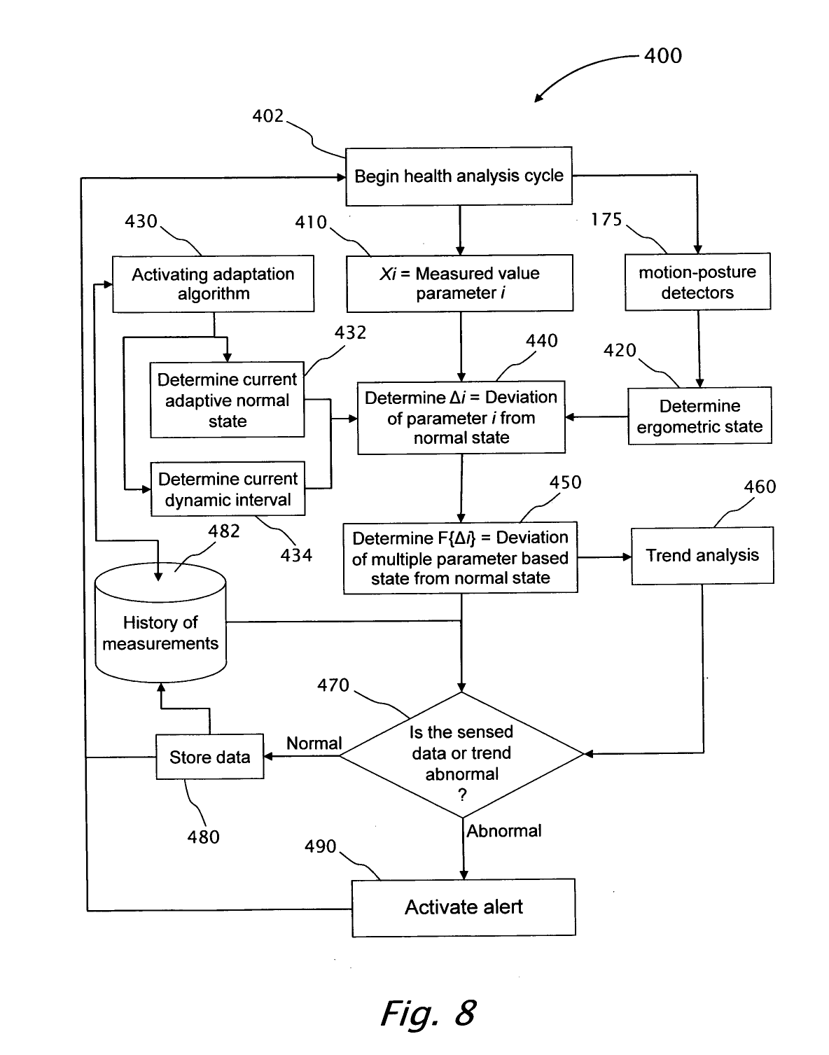

Reference is now made to FIG. 8, which is a schematic flow diagram that outlines a cycle 400 of monitoring the health status of a living being, according to variations of the present invention. Cycle 400 begins in virtual step 402 and proceeds in the following steps:
Step 410: sensing designated health related parameter, thereby generating sensed data.
   Each sensor, senses the parameter that a sensor, is designed to measure, and thereby generating sensed data Xi.
Step 420: determine ergometric state.
   The ergometric state of the monitored living being is determined, that is the motion state and the bodily orientation of the monitored living being.
Step 430: perform data analysis using adaptation algorithm.
   Analysis center 120 activates an adaptation algorithm to compute the following:
   Step 432: determine the current adaptive normal state
      Determine the current normal state of the monitored living being, adjusted to a variety of personal parameters of the monitored living being. The History of measurements of the monitored living being is obtained from database 482.
   Step 434: determine the current dynamic interval.
      Determine the current dynamic interval of the monitored living being, forming the envelope in which the health state of the monitored living being is considered normal and out of which the health state of the monitored living being is considered abnormal. The History of measurements of the monitored living being is obtained from database 482.
Step 440: determine the deviation of the measured value Xi from normal state.
   The deviation Δi of the measured value Xi from normal state is determined.
Step 450: determine the deviation of a group of measured values, from normal state.
   The deviation F{Δi} a group of measured values from normal state is determined.
Step 460: perform trend analysis.
   A trend analysis performed to compute the deviation a trend from normal state.
Step 470: determining if the sensed data or trend is abnormal.
   If the sensed data or trend is determined to be abnormal, go to step 490.
Step 480: store data.
   Store all sensed data and computed data in Database 482.
   Go to step 402.
Step 490: activate alert.
   The sensed data or trend is determined to be abnormal and therefore, the alerting unit (150 or 250) is activated.
   Go to step 402.
(end of steps details of cycle 400)

It should be noted that health monitoring and alert system (100, 200) is designed to detect one or more potentially health hazardous situations.

Preferably, health monitoring and alert system (100, 200) complies with to the IEEE 802.15 standard (currently under planning) and FCC Medical Body Area Network (MBAN) systems (currently under planning).

It should be further noted that the monitoring of the health condition is done continuously. Alerts are generated immediately as a dangerous situation is detected. The user does not have to perform any activity action in order to get the alert. For the sake of clarity, activity may be required at installation time, but not during monitoring.

It should be further noted that alerts can be issued to the monitored being and/or to an external entity, such as an emergency center, a close relative, etc. The alert can be transmitted to a computer, a telephone and/or any other communication device.

It should be further noted that the health monitoring and alert system can optionally send the data to any remote processor, which can further process the information, compare it to many other monitored people, make statistics-based decisions and other decision-making methods to improve alerts and providing information for the treatment of the living being once getting to a treating facility.

The invention being thus described in terms of embodiments and examples, it will be obvious that the same may be varied in many ways. Such variations are not to be regarded as a departure from the spirit and scope of the invention, and all such modifications as would be obvious to one skilled in the art are intended to be included within the scope of the following claims.

1. A seamless health monitoring and alerting system, configured for use by both a healthy and an unhealthy living being, comprising:
a) a control module including:
i) an analysis subsystem having a processing unit; and
ii) an alerting unit;
b) a communication unit; and
c) one or more sensors,
wherein each of said one or more sensors is configured to detect a predetermined physiological or chemical parameter of said living being;
wherein said communication unit is facilitated to receive said detected parameters from each of said one or more sensors and transmit said detected parameters to said analysis subsystem;
wherein said processor of said analysis subsystem of said control module analyzes said detected parameters to thereby determine if one or more of said detected parameters or a combination of said detected parameters is abnormal;
wherein, when at least one of said detected parameters, or a combination thereof, is determined to be abnormal, said alerting unit is operatively activated by said processing unit to alert said living being carrying the health monitoring system and optionally, one or more predetermined alert receiving entities; and
wherein, when said alerting unit is activated to alert said living being, carrying the seamless health monitoring system, the health monitoring and alert system is fully independent of any remote entity.

2. (canceled)

3. The health monitoring system as in claim 1, wherein said analysis subsystem includes a memory for storing at least a portion of said detected parameters; wherein said stored detected parameters are used for trends analysis, for adaptation analysis, for abnormality prediction analysis, and for enabling data extraction for further external analysis; and wherein said analysis subsystem analyzes said trends to detect abnormal trends in said detected parameters or a combination thereof, and wherein, when at least one of said trends is determined to be abnormal, said alerting unit is operatively activated to alert one or more predetermined alert receiving entities.

4. (canceled)

5. The health monitoring system as in claim 1, wherein said health monitoring system is facilitated to operate substantially continuously.

6. (canceled)

7. The health monitoring system as in claim 1, wherein the boundary definition of said parameter abnormality is personally, dynamically and automatically adapted to the changes over time of the normal state of said living being.

8. (canceled)

9. (canceled)

10. The health monitoring system as in claim 1, wherein the system is facilitated to provide said alert with no operative action required to be performed by said living being during the health monitoring.

11. The health monitoring system as in claim 1, wherein said control module is selected form the group consisting of a wearable module, an in-vivo module and a mobile device.

12. (canceled)

13. The health monitoring system as in claim 1, wherein said one or more sensors is selected from the group of sensors consisting of a wearable sensor and an in-vivo sensor.

14. (canceled)

15. The health monitoring system as in claim 13, wherein said in-vivo sensor is a nano-sensor, and wherein said nano-sensor is delivered to an in-vivo target location, via blood vessels or delivered to a subcutaneous location.

16. (canceled)

17. (canceled)

18. (canceled)

19. (canceled)

20. The health monitoring system as in claim 15, wherein said in-vivo sensor is delivered to an in-vivo target location, in a non-painful laser-based insertion, RF-based technology, by swallowing pills or capsules, by trans-dermal delivery patches, by injection using micro-needles, by nano-needles, using targeted-liposome delivery techniques, or by using nanotube delivery techniques.

21. (canceled)

22. (canceled)

23. (canceled)

24. The health monitoring system as in claim 15, wherein said communication unit or said communication subunit are configured to transmit a signal to said in-vivo sensor and to receive a modulated echo of said transmitted signal, returned from said in-vivo sensor, and wherein said modulated echo carries the information sensed by said in-vivo sensor, thereby eliminating the need of a power source for said nano-sensor.

25. (canceled)

26. The health monitoring system as in claim 11, wherein said wearable control unit is a seamless wearable device attached to, or integrated into or a software product embodied in a non-transitory mobile electronic device, a wheel chair or a personal device carried regularly by said living being.

27. (canceled)

28. The health monitoring system as in claim 1, wherein said control module is disposed in the oral cavity of said living being, thereby being an oral control unit, and wherein preferably, said oral control unit is disposed inside one or more natural or artificial teeth of said living being.

29. (canceled)

30. The health monitoring system as in claim 28, wherein said oral control unit further comprises:
iii) an internal power source; and
iv) a maintenance unit,
wherein said maintenance unit facilitates external maintenance activities of said oral control unit; and
wherein said external maintenance activities are selected from a group of maintenance activities including updating said processing unit, downloading data, setup and update of parameters, insert a new or a replacement nano sensor, loading test analysis elements, unloading disposal waste, recharging internal power source, and perform diagnostic processes of selected members of the health monitoring system.

31. (canceled)

32. The health monitoring system as in claim 30, wherein said oral control unit further comprises:
v) an oral test unit having at least one oral sensor; and
vi) at least one oral sampler,
wherein said maintenance unit preferably includes a storage for test analysis elements and a storage for waste resulting from tests analysis;
wherein said at least one oral sampler collects oral material selected from the group of orally disposed materials including oral fluids, breath and blood;
wherein said oral test unit is operatively activated to engage one or more of said analysis elements with said one or more oral materials, thereby producing a testable material;
wherein said at least one oral sensor is configured to sense said testable material and create tested data; and
wherein said maintenance unit transfers said tested data to said analysis subsystem of said control module.

33. (canceled)

34. The health monitoring system as in claim 30, wherein said maintenance unit includes storage for nano sensors and/or reagents, and wherein said maintenance unit is facilitated to deliver said nano sensor and/or reagent on demand or by a predetermined time interval.

35. The health monitoring system as in claim 30, wherein said maintenance unit further includes a loading service channel for loading said storage for test analysis elements from an external source, wherein said loading service channel is sealingly closed during normal operation; and a disposing service channel for disposing accumulated waste from said storage for waste to an external location, wherein said disposing service channel is sealingly closed during normal operation.

36. (canceled)

37. (canceled)

38. (canceled)

39. (canceled)

40. (canceled)

41. The health monitoring system as in claim 1, wherein said one or more sensors is selected from the group of physical sensors including an electric sensor, an optical sensor, an acoustic sensor, an acceleration sensor, a pressure based sensor, an impedance sensor, a conductivity sensor and a humidity sensor.

42. The health monitoring system as in claim 41, wherein said physical sensors, such as accelerometers, to sense bodily motion and posture related data, to thereby facilitate freedom of motion to the carrying living being.

43. (canceled)

44. (canceled)

45. (canceled)

46. (canceled)

47. (canceled)

48. (canceled)

49. A method for monitoring the health status of a living being, comprising the steps of:
a) providing a seamless health monitoring and alerting system, configured for use by the healthy living being, the system including:
i) a control module having:
(A) an analysis subsystem having a processing unit; and
(B) an alerting unit;
ii) a communication unit; and
iii) one or more sensors;
b) sensing at least one designated health related parameter by said one or more sensors, thereby generating sensed data;
c) transmitting said sensed data to said communication unit;
d) transmitting said sensed data to said analysis unit;
e) analyzing said sensed data;
f) determining if said sensed data is abnormal; and
g) upon determining that said sensed data is abnormal,
i) selecting a proper alert type;
ii) transmitting said selected alert type(s) to said alerting unit; and
iii) activating said alerting unit by said processing unit, to thereby alert said living being carrying the health monitoring system and optionally, one or more predetermined alert receiving entities.

50. The method as in claim 49 further including the steps of:
h) analyzing said sensed data by said processing unit, thereby generating analyzed sensed data;
i) determining if said sensed analyzed data is abnormal;
j) upon determining that said analyzed sensed data is abnormal,
i) selecting a proper alert type;
ii) transmitting said selected alert type(s) to said alerting unit; and
iii) activating said alerting unit.

51. The method as in claim 49, wherein said control module includes an oral control unit, disposed in the oral cavity of said living being, wherein said oral control unit further includes a maintenance unit, wherein said seamless health monitoring system further includes an oral test unit having at least one oral sensor, and at least one oral sampler, and wherein the method further includes the steps of:
h) collecting selected oral materials from said oral cavity of the living being;
i) transferring said oral materials to said test unit;
j) transferring test analysis elements from an optional storage for test analysis elements or from external sources, to said test unit;
k) engaging said analysis elements with said oral materials, thereby producing a testable material;
l) sensing said testable material by said at least one oral sensor, thereby creating sensed data; and
m) proceeding with step (d) of the method.

52. (canceled)

53. (canceled)

54. (canceled)

55. (canceled)

56. (canceled)

57. (canceled)

58. The method as in claim 49 further including the steps of:
h) analyzing said sensed data by said processing unit, thereby generating trend analyzed data;
i) determining if said trend analyzed data is analyzed to predict a future abnormal health state;
j) upon predicting a future abnormal health state,
i) selecting a proper alert type;
ii) transmitting said selected alert type(s) to said alerting unit; and
iii) activating said alerting unit.
